(19) United States
(12) Patent Application Publication
(10) Pub. No.: US 2004/0146878 A1
(43) Pub. Date: Jul. 29, 2004

(54) Method for gene diagnosis of bovine Hsp70 deficiency

(75) Inventors: Mayumi Sugimoto, Nishishirakawa-gun (JP); Hidefumi Furuoka, Makubetsu-cho (JP); Yoshikazu Sugimoto, Nishishirakawa-gun (JP)

Correspondence Address: FRISHAUF, HOLTZ, GOODMAN & CHICK, PC, 767 THIRD AVENUE, 25TH FLOOR, NEW YORK, NY, 10017-2023, US

(73) Assignee: Director of Livestock Improvement Association of Japan, Inc., Tokyo (JP)

(21) Appl. No.: 10/609,181

(22) Filed: Jun. 26, 2003

(30) Foreign Application Priority Data
Nov. 12, 2002 (JP) 2002-327856

(51) Int. Cl. C12Q 1/68; C12P 19/34

(52) U.S. Cl. 435/6; 435/91.2

(57) ABSTRACT

A method for gene diagnosis of bovine Hsp70 deficiency, which comprises (a) a step of obtaining a bovine nucleic acid sample, (b) a step of subjecting the nucleic acid sample obtained in step (a) to a gene amplification reaction to obtain a nucleic acid fragment in which a region including a mutation site likely to be present in bovine Hsp70 gene is amplified, and (c) a step of examining the presence of mutation on the nucleic acid fragment in step (b), the region including the mutation site being a region including 1997-11030 position of a base sequence shown in SEQ ID No. 1 of SEQUENCE LISTING in a base sequence of bovine Hsp70 gene. According to the invention, bovine Hsp70 deficiency and its carrier can be detected and diagnosed by a genetic engineering method easily and quickly, and a kit used for this purpose is provided.

### FIELD OF THE INVENTION

The present invention relates to a method for gene diagnosis (or detection) of bovine Hsp70 deficiency.

In the invention, “Hsp70” means a protein formed by transcribing and translating Hsp70 gene, and “Hsp70 gene” means a DNA region including an exon of a translated region encoding Hsp70, an exon of an adjacent untranslated region, introns of these regions, a region participating in control of expression of the gene and a mutant portion related with the disease.

### BACKGROUND OF THE INVENTION

The Hsp70 deficiency is an autosomal recessive hereditary disease which is mainly a diaphragmatic muscle disease, clinically showing tympania, respiratory insufficiency and the like. In this disease, muscle fiber denaturation and a core-like structure are observed in a diaphragmatic muscle as histopathological characteristics. The attack of this disease has been found in a Holstein since 1994, while no such disease is reported in humans.

Since bovine attacked with Hsp70 deficiency repeats tympania, the disease can be found. However, with respect to bovine being a heterozygote carrying a gene related with abnormality only on one chromosome, namely bovine being genetically a carrier of Hsp70 deficiency, it is difficult to find the abnormality.

Accordingly, even in case of mating a bovine pair with abnormality unobserved apparently, abnormality sometimes appears in a calf to be born. Thus, such a case is problematic in view of preventing attack of the disease.

In order to prevent bovine Hsp70 deficiency, a method in which mating of heterozygotes is avoided is considered. However, for enabling this method, it is required that diagnosis for bovine Hsp70 deficiency is performed at a gene level to specify a carrier of a disease gene. If it is possible to provide a method which can clarify how a bovine disease gene having abnormality is mutated in comparison to a normal bovine gene and rapidly detect a mutant gene by various genetic engineerings, the genetic diagnosis can be established.

### SUMMARY OF THE INVENTION

Accordingly, it is an object of the invention to provide a method for gene diagnosis (detection) of bovine Hsp70 deficiency. This makes it possible to prevent attack of the disease in future by screening a carrier of Hsp70 deficiency.

The present inventors have conducted investigations for achieving the foregoing object, and have consequently succeeded in finding deficiency of DNA ranging approximately 11 kb closely related with the disease. The deficient region includes Hsp70 gene already reported as to bovine (M. D. Groz et al., Genomics, 14, 863-868 (1992); J. A. Gutierrez et al., Biochem. J., 305, 197-203 (1995)).

Further, the present inventors have found that the cause of this disease is that Hsp70 is not expressed owing to mutation, i.e. DNA deficiency, and that the mutation is detected by PCR (polymerase chain reaction) using oligonucleotide primers including specific oligonucleotide primers set in the gene. These findings have led to the completion of the invention.

The invention and the embodiments thereof are described below.

The invention is described below.

(1) A method for gene diagnosis of bovine Hsp70 deficiency, which comprises the following steps,

(a) a step of obtaining a bovine nucleic acid sample,

(b) a step of subjecting the nucleic acid sample obtained in step (a) to a gene amplification reaction to obtain a nucleic acid fragment in which a region including a mutation site likely to be present in bovine Hsp70 gene is amplified, and

(c) a step of examining the presence of mutation in the nucleic acid fragment in step (b),

the region including the mutation site being a region including 1997-11030 position of a base sequence shown in SEQ ID No. 1 of SEQUENCE LISTING in a base sequence of bovine Hsp70 gene.

(2) The method for gene diagnosis described in (1), wherein the gene amplification reaction is conducted by a polymerase chain reaction method.

(3) The method for gene diagnosis described in (1) or (2), wherein the presence of mutation is examined by examining a gene amplification product obtained by the polymerase chain reaction method.

(4) The method for gene diagnosis described in any one of (1) to (3), wherein the nucleic acid sample is a sample containing genomic DNA, cDNA or mRNA.

(5) A method for gene diagnosis of bovine Hsp70 deficiency, which comprises conducting genome linkage analysis of subject bovine, isolating bovine Hsp70 gene by positional cloning, determining a base sequence of the gene by a usual manner, and examining the presence or absence of mutation by comparing said base sequence with a base sequence of cDNA encoding normal bovine Hsp70 as shown in SEQ ID No. 1 of SEQUENCE LISTING.

(6) A kit for detecting bovine Hsp70 deficiency, which kit contains oligonucleotide primers used for amplifying a region including a mutation site likely to be present in bovine Hsp70 gene by a gene amplification reaction, the oligonucleotide primers being selected from the group consisting of

<1> oligonucleotides having a base sequence corresponding to a 5′-terminal region in a base sequence shown in SEQ ID No. 1 of SEQUENCE LISTING, and

<2> oligonucleotides having a complementary base sequence to a 3′-terminal region in the base sequence shown in SEQ ID No. 1 of SEQUENCE LISTING.

(7) The kit described in (6), wherein the oligonucleotide primers comprise from 15 to 35 nucleotides.

(8) The kit described in (6), wherein the oligonucleotide primers are a pair of oligonucleotide primers selected from the group consisting of those shown in SEQ ID Nos. 2 to 8 of SEQUENCE LISTING, provided combinations of SEQ ID Nos. 2 and 4, 3 and 5, and 6 and 7 are excluded.

(9) A base sequence which is a whole or a part of a base sequence corresponding to bovine Hsp70 gene or bovine Hsp70 deficiency gene or its complementary chain, and which is selected from the group consisting of

(a) a whole or a part of a base sequence shown in SEQ ID No. 1 of SEQUENCE LISTING or its complementary chain,

(b) all sequences which can be used for hybridization to the sequence (a) and gene amplification of a region including a mutation site likely to be present in bovine Hsp70 gene by PCR, and

(c) sequences derived from the sequences (a) and (b) for degeneracy of a gene code.

(10) The base sequence described in (9), which is selected from the group consisting of a genomic DNA sequence, a cDNA sequence, an RNA sequence, a hybrid sequence, a synthetic sequence and a semi-synthetic sequence.

(11) A nucleotide probe which can be hybridized to the base sequence described in any of (9) and (10) or the corresponding mRNA.

(12) A method for detection of bovine Hsp70 deficiency, which comprises a step of clarifying and/or isolating a sequence including a mutation site likely to be present in bovine Hsp70 gene using the nucleotide probe described in (11).

(13) The oligonucleotide primers described in any of (6) to (8).

(14) A gene diagnostic reagent for bovine Hsp70 deficiency, containing the oligonucleotide primers described in (13).

### BRIEF DESCRIPTION OF THE DRAWINGS

FIG. 1(a) is a view showing a deficient portion of bovine Hsp70 gene derived from Hsp70 deficiency-attacked bovine, and FIG. 1(b) is a view showing positions of PCR primers for detecting the deficient portion.

FIG. 2(a) is an electrophoresis pattern view showing amplification by PCR using genomic DNA free from mutation of Hsp70 as a template for detection of Hsp70 normal type, and FIG. 2(b) is an electrophoresis pattern view showing amplification by PCR using genomic DNA with mutation of Hsp70 as a template for detection of Hsp70 mutant type.

### DETAILED DESCRIPTION OF THE INVENTION

The invention is described in detail below.

The invention described in (1) is a method for gene diagnosis (detection) of bovine Hsp70 deficiency.

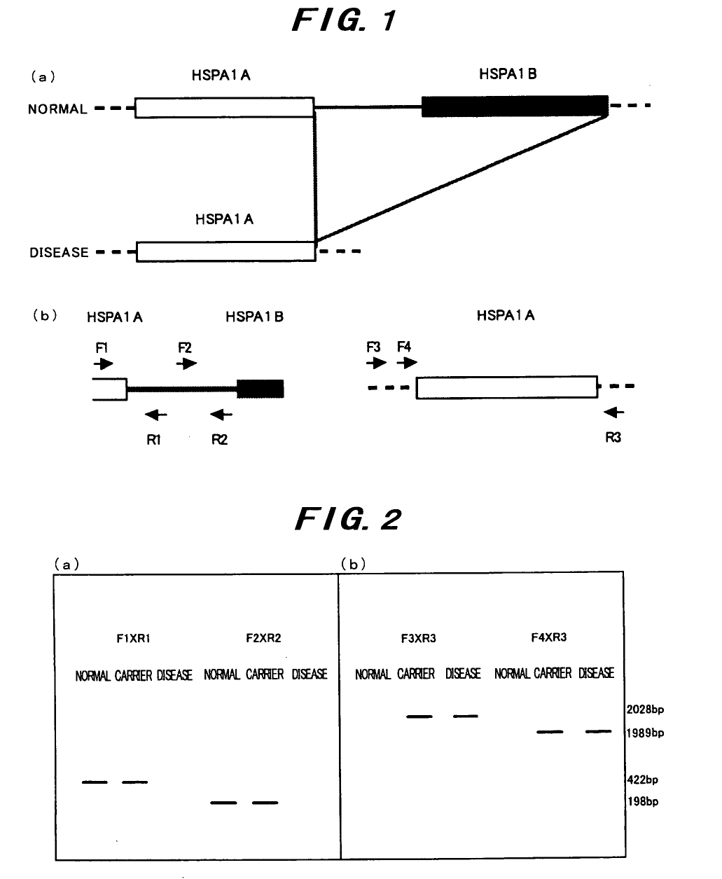

As will be later described, when the mutation of gene which causes bovine Hsp70 deficiency is clarified (FIG. 1(a)) the detection of the disease can be performed using this mutation. The specific method for gene diagnosis (detection) of bovine Hsp70 deficiency comprises the following steps,

(a) a step of obtaining a bovine nucleic acid sample,

(b) a step of subjecting the nucleic acid sample obtained in step (a) to a gene amplification reaction to obtain a nucleic acid fragment in which a gene including a mutation site likely to be present in bovine Hsp70 gene is amplified, and

(c) a step of examining the presence of mutation in the nucleic acid fragment in step (b).

First, step (a) is described. The bovine nucleic acid sample used in the invention is not particularly limited so long as it has a base sequence encoding Hsp70. It includes nucleic acids derived from appropriate cells or tissues (including cDNAs transcribed from all genomic DNAs and all RNAs of cells), such as genomic DNAs, cDNAs and mRNAs. The bovine nucleic acid sample can be prepared by a known method, for example, the method described in Molecular Cloning, A Laboratory Manual (2nd edition) (J. Sambrook, E. F. Fritsch & T. Maniatis (Ed.), Cold Spring Harbor Press, Cold Spring Harbor, N.Y. (1989)).

Second, step (b) is described. The region including the mutation site likely to be present in bovine Hsp70 gene is amplified by using the nucleic acid sample obtained in step (a) and appropriate primers, whereby a desired nucleic acid fragment can be obtained.

The method for the gene amplification reaction used in this step is not particularly limited so long as it can amplify the very region. For example, an PCR method, a nucleic acid amplification method using RNA polymerase and a chain substitution amplification method can be used. Of these, the PCR method is preferable.

The mutation site-including region to be amplified is not particularly limited so long as it includes mutation that causes bovine Hsp70 deficiency in the base sequence of bovine Hsp70 gene. For example, a region including 1997-11030 position in the base sequence shown in SEQ ID No. 1 of SEQUENCE LISTING is mentioned.

In the present specification, “polymerase chain reaction” or “PCR” generally refers to the method described in U.S. Pat. No. 4,683,195, for example, a method for enzymatically amplifying in vitro a desired base sequence.

In general, the PCR method includes repeating a cycle of primer elongation (primer extension and synthesis) by using two oligonucleotide primers capable of being hybridized to a template nucleic acid preferentially. As typical primers used in the PCR method, primers complementary to a base sequence to be amplified in a template are available. For example, primers complementary to the base sequence to be amplified at both ends or primers adjacent to the base sequence to be amplified are preferably used.

PCR can be conducted by the method described in M. A. Innis, D. M. Gelfaud, J. J. Snindky, & T. J. White (Ed.), PCR protocols: A Guide To Methods And Applications, Academic Press, Inc., New York (1990); M. J. McPherson, P. Quirke & G. R. Taylor (Ed.), PCR: A Practical Approach, IRL Press, Oxford (1991); R. K. Saiki et al., Science, 239, 487-491 (1988); M. A. Frohman et al., Proc. Natl. Acad. Sci. USA, 85, 8998-9002 (1988), or its modified or altered method.

Further, the PCR method can be performed by using an appropriate commercial kit and also according to protocols given by kit makers or distributors.

In the present specification, “oligonucleotide” is a relatively short single-stranded or double-stranded chain polynucleotide. Polydeoxynucleotide is preferable, and it can chemically be synthesized by a known method such as a triester method, a phosphite method, a phosphoamidite method or a phosphonate method as described in Agnew. Chem. Int. Ed. Engl., Vol. 28, pp. 716-734 (1989). It is commonly known that the synthesis can conveniently be conducted on a modified solid support. For example, it can be conducted with an automated synthesizer which is commercially available. The oligonucleotide may contain one or more modified bases. For example, it may contain a base unusual in nature, such as inosine, or a tritylated base.

The primers used in the PCR method are not particularly limited so long as they can amplify the DNA fragment including the mutation site. As typical primers, (a) oligonucleotides having a base sequence corresponding to an optional region in a base sequence shown in SEQ ID No. 1 of SEQUENCE LISTING and

(b) oligonucleotides having a complementary base sequence to an optional region in the base sequence shown in SEQ ID No. 1 of SEQUENCE LISTING can be used. Preferably, (1) oligonucleotides having a base sequence corresponding to a 5′-terminal optional region in the base sequence shown in SEQ ID No. 1 of SEQUENCE LISTING and (2) oligonucleotides having a complementary base sequence to a 3′-terminal optional region in the base sequence shown in SEQ ID No. 1 of SEQUENCE LISTING can be used. Examples thereof include oligonucleotides containing from 3 to 100, preferably from 10 to 50, more preferably 15 to 35 nucleotides.

The PCR conditions are not particularly limited either, and known PCR conditions are available. The conditions can be selected by referring to the descriptions of the foregoing documents. In PCR, a cycle of thermal denaturation of DNA chain, annealing of primers and synthesis of complementary chain with polymerase is repeated, for example, from 10 to 50 times, preferably from 20 to 35 times, more preferably from 25 to 30 times.

Third, step (c) is described. In this step, the presence of mutation is examined on the nucleic acid fragment obtained in step (b). The method for detecting the presence of mutation is not particularly limited, and the presence of mutation is detected by examining a length of the DNA fragment obtained by the PCR method.

A method for examining the length of the DNA fragment is not particularly limited. Preferable is a method in which a DNA fragment is separated by electrophoresis on a polyacrylamide or agarose gel and the desired DNA fragment is identified on the basis of, for example, its mobility relative to a mobility of a marker DNA fragment having a known molecular weight.

As a detecting method other than the above method, there is a method in which the foregoing step (c) is replaced with another mutation detecting method. For detecting the mutation, a known method for detecting mutation, such as a hybridization method using an appropriate DNA fragment including a mutation site as a probe, can be used. Moreover, the mutation can be detected by a method in which a base sequence is determined by cloning an amplified DNA in an appropriate vector or a method in which by using an amplified fragment per se as a template, its base sequence is determined.

It is advisable that oligonucleotides or a probe is labeled with a label component for facilitating the detection. The label component can be detected by a spectroscopic method, an optical method, a biochemical method, an immunological method, an enzyme chemical method or a radiochemical method. Examples of the label component include enzymes such as peroxidase and alkaline phosphatase, radioactive labels such as ³²P, isotopes, biotin, fluorochrome, luminous substances and coloring matters.

It is possible to detect and diagnose not only bovine attacked with Hsp70 deficiency but also bovine being a carrier of Hsp70 deficiency by the method for gene diagnosis (detection) of bovine Hsp70 deficiency of the invention.

Accordingly, the bovine Hsp70 deficiency of the invention means the genetic abnormality, and it is interpreted in a broad sense by including a carrier, regardless of the presence or absence of the disease.

Analysis of Hsp70 gene of normal bovine and Hsp70 deficiency-attacked bovine:

For examining a relation between mutation in a gene and the disease, first, a gene (cDNA) encoding normal bovine Hsp70 is isolated, and its base sequence is clarified. Any example of isolating the very gene has not been reported as yet. The present inventors performed genome linkage analysis of a family including the disease-attacked bovine, and isolated bovine Hsp70 gene by a positional cloning method. That is, it was performed by mapping a causal gene locus on a linkage map and then isolating the causal gene from the chromosomal region (“Dobutsu Iden Ikushugaku Jiten”, published by Japan Livestock Technology Association).

The whole base sequence of cDNA encoding normal bovine Hsp70 clarified in the invention is shown in SEQ ID No. 1 of SEQUENCE LISTING. Determination of the base sequence of the DNA fragment (sequencing) can be performed by a chemical analysis method (Maxam & Gilbert method) or a chain terminator method (Sanger dideoxy method).

The mutation in the gene that causes Hsp70 deficiency can be clarified by comparing base sequences of Hsp70 genes of normal bovine and attacked bovine. That is, the mutation that causes the disease can be identified by examining the base sequence of attacked bovine Hsp70 gene as in the normal bovine and comparing it with the base sequence of the normal bovine gene.

The mutation that a portion of 1997-11030 position in the base sequence of the translated region encoding Hsp70 in normal bovine Hsp70 gene shown in SEQ ID No. 1 of SEQUENCE LISTING is deficient in Hsp70 gene of Hsp70 deficiency-attacked bovine has been found in the invention (FIG. 1(a)).

In FIG. 1(a), both HSPA1A and HSPA1B are Hsp70 genes. The Hsp70 genes are two genes of almost the same sequences which are laterally arranged on a chromosome. Further, the 11-kb deficient site starts from the 3′-untranslated region of HSPA1A and ends with the 3′-terminal translated region of HSPA1B.

According to the invention, bovine Hsp70 deficiency and its carrier can be detected and diagnosed by a genetic engineering method easily and quickly. Further, a kit used for this purpose is provided.

### EXAMPLES

The invention is illustrated more specifically below by referring to Examples. However, the invention is not limited thereto. In the following description, the operation is performed by a method described in D. M. Glover & B. D. Hames (Ed.), DNA Cloning 1, Core Techniques (2nd edition), A Practical Approach, Oxford University Press, Oxford (1995); J. Sambrook, E. F. Fritsch & T. Maniatis (Ed.), Molecular Cloning, A Laboratory Manual (2nd edition), Cold Spring Harbor Press, Cold Spring Harbor, N.Y. (1989); M. A. Innis, D. M. Gelfaud, J. J. Snindky & T. J. White (Ed.), PCR Protocols: A Guide To Methods and Applications, Academic Press, Inc., New York (1990); M. J. McPherson, P. Quirke & G. R. Taylor (Ed.), PCR; A Practical Approach, IRL Press, Oxford (1991), or its modified or altered method, unless otherwise instructed. When a commercial kit or measuring device is used, the operation is performed according to instructions of protocols attached thereto, unless otherwise instructed.

### Example 1

(1) Determination of a Base Sequence of Normal Bovine Hsp70 Gene

With respect to a family consisting of Hsp70 deficiency-attacked bovine (12 heads) and parents/daughter bovine thereof, a region of bovine chromosome linked with Hsp70 deficiency was determined using a polymorphic DNA marker. That is, a DNA marker linked most strongly with the disease among DNA markers present on a linkage map was selected. A bovine BAC (bacterial artificial chromosome) clone present in this region was separated from BAC library [manufactured by CHILDREN'S HOSPITAL OAKLAND—BACPAC RESOURCES].

Shotgun base sequence determination was performed using this BAC clone as a material. First, DNA of the BAC clone was physically cut to a size of approximately 700 bp with a nebulizer, and both ends were blunted with DNA polymerase. The cut fragments were cloned in a plasmid. Arbitrary selection was performed from these plasmid clones, and the base sequence of each of the selected clones was determined from both ends with BigDye Terminator Cycle Sequencing Reagent (manufactured by PE Biosystems) using a 3700 fluorescent DNA sequencer (manufactured by PE Biosystems). The resulting base sequence is shown in SEQ. ID No. 1 of SEQENCE LISTING. This base sequence was compared with another known gene (gene with the base sequence determined as registered in database of GenBank). Consequently, it was found to be Hsp70 gene.

(2) Expression of Hsp70

A soluble fraction as a supernatant obtained by centrifugation of a mashed muscle was prepared from a diaphragmatic muscle of deficiency-attacked bovine, and the expression of Hsp70 was examined in a usual manner. That is, western blotting was performed such that the soluble fraction was subjected to electrophoresis on a 10%-polyacrylamide gel containing SDS and blotted on a nitrocellulose membrane and expression of Hsp70 was examined using an anti-Hsp70 antibody (manufactured by Santa Cruz Biotechnology). Consequently, it was found that Hsp70 was little expressed in deficiency-attacked bovine in comparison to normal bovine.

(3) Determination of a Base Sequence of Hsp70 Gene of Hsp70 Deficiency-Attacked Bovine

As described in (2) of Example 1, Hsp70 was little expressed in Hsp70 deficiency-attacked bovine, and the base sequence of Hsp70 gene in deficiency-attacked bovine was determined from this fact. That is, PCR was performed by referring to the sequence of SEQ. ID No. 1 of SEQUENCE LISTING to determine the DNA base sequence of Hsp70 gene in Hsp70 deficiency-attacked bovine.

The thus-determined base sequence was compared with the foregoing base sequence determined in normal bovine. Consequently, in Hsp70 gene of Hsp70 gene deficiency-attacked bovine, approximately 11 kb including 1997-11030 position was deficient in the base sequence shown in SEQ ID No. 1 of SEQUENCE LISTING. For this reason, it is considered that the expression of Hsp70 is not observed in deficiency-attacked bovine.

### Example 2

Detection of Hsp70 Normal Type:

In order to identify a normal type of Hsp70 in bovine genomic DNA, primers F1, R1, F2 and R2 (SEQ ID Nos. 2 to 5 of SEQUENCE LISTING) for amplifying a DNA fragment including a deficient site found in Hsp70 deficiency-attacked bovine were synthesized according to the base sequence of SEQ. ID No. 1 of SEQUENCE LISTING. The positions of these primers in the base sequence are shown in FIG. 1(b).

Genomic DNAs were prepared from blood of normal bovine, muscle of Hsp70 deficiency-attacked bovine and blood of mother or daughter bovine thereof (containing EDTA and heparin as an anticoagulant) using QIAamp Blood Kit or QIAamp Tissue Kit (manufactured by QIAGEN).

PCR was conducted using these genomic DNAs as templates, primers F1 and R1, and primers F2 and R2, and Animal Taq. In PCR, a cycle including steps at 94° C. for 20 seconds, at 60° C. for 30 seconds and at 72° C. for 1 minute was repeated 35 times. The reaction solution was subjected to electrophoresis using a 1.5% gel (1×TBE), and the gel after the electrophoresis was stained with ethidium bromide to identify the amplified DNA fragments.

When the genomic DNAs of normal bovine and mother bovine or daughter bovine of Hsp70 deficiency-attacked bovine were used as templates, the amplifications of 422-bp and 198-bp DNA fragments were observed as shown in FIG. 2(a). However, when the genomic DNA of Hsp70 deficiency-attacked bovine was used as a template, no amplification was observed.

### Example 3

Detection of Hsp70 Mutant Type:

In order to identify the presence of Hsp70 gene mutant type in bovine genomic DNA, primers F3, F4 and R3 (SEQ ID Nos. 6 to 8 of SEQUENCE LISTING) were synthesized (FIG. 1(b)) on both ends of an approximately 11-kb deficient portion described in (3) of Example 1, and subjected to experiment.

PCR was conducted using genomic DNAs of normal bovine, Hsp70 deficiency-attacked bovine and mother bovine or daughter bovine thereof as templates, primers F3 and R3, and primers F4 and R3, and Animal Taq. In PCR, a cycle including steps at 94° C. for 20 seconds, at 60° C. for 30 seconds and at 72° C. for 1 minute was repeated 35 times. The reaction solution was subjected to electrophoresis using a 1.5% gel (1×TBE), and the gel after the electrophoresis was stained with ethidium bromide to identify the amplified DNA fragments.

When the genomic DNAs of Hsp70 deficiency-attacked bovine and mother bovine or daughter bovine thereof were used as templates, the amplifications of 2028-bp and 1989-bp DNA fragments were observed as shown in FIG. 2(b). However, when the genomic DNA of normal bovine was used as a template, no amplification was observed.

These results reveal that with respect to the deficiency of approximately 11 kb including the translated region of Hsp70 gene, the mother or daughter bovine of the deficiency-attacked bovine is a heterozygote to this mutation and the Hsp70 deficiency is a hereditary disease with autosome recessive heredity.

### Sequence Listing

<110> Japan Livestock Technology Association

<120> Gene Diagnosis for Bovine Hsp70 Deficiency

<130> P141292K

<160>8

<210>1

<211>12988

<212>DNA

<213>Bovine

<400>1

acgtcgttga tcctgtgggc cgttttcagg tttgaagctt atctcggagc cgaaaaggca 60

gggcaccggc atggcgaaaa acatggctat cggcatcgac ctgggcacca cctactcctg 120

cgtaggggtg ttccagcacg gcaaggtgga gatcatcgcc aacgaccagg gcaaccgcac 180

cacccccagc tacgtggcct tcaccgatac cgagcggctc atcggcgatg cggccaagaa 240

What is claimed is:

1. A method for gene diagnosis of bovine Hsp70 deficiency, which comprises the following steps,
(a) a step of obtaining a bovine nucleic acid sample,
(b) a step of subjecting the nucleic acid sample obtained in step (a) to a gene amplification reaction to obtain a nucleic acid fragment in which a region including a mutation site likely to be present in bovine Hsp70 gene is amplified, and
(c) a step of examining the presence of mutation on the nucleic acid fragment in step (b),
the region including the mutation site being a region including 1997-11030 position of a base sequence shown in SEQ ID No. 1 of SEQUENCE LISTING in a base sequence of bovine Hsp70 gene.

2. The method for gene diagnosis as claimed in claim 1, wherein the gene amplification reaction is conducted by a polymerase chain reaction method.

3. The method for gene diagnosis as claimed in claim 1 or 2, wherein the presence of mutation is examined by examining a gene amplification product obtained by the polymerase chain reaction method.

4. The method for gene diagnosis as claimed in any one of claims 1 to 3, wherein the nucleic acid sample is a sample containing genomic DNA, cDNA or mRNA.

5. A method for gene diagnosis of bovine Hsp70 deficiency, which comprises conducting genome linkage analysis of subject bovine, isolating bovine Hsp70 gene by positional cloning, determining a base sequence of the gene by a usual manner, and examining the presence or absence of mutation by comparing said base sequence with a base sequence of cDNA encoding normal bovine Hsp70 as shown in SEQ ID No. 1 of SEQUENCE LISTING.

6. A kit for detecting bovine Hsp70 deficiency, which kit contains oligonucleotide primers used for amplifying a region including a mutation site likely to be present in bovine Hsp70 gene by a gene amplification reaction, the oligonucleotide primers being selected from the group consisting of
(1) oligonucleotides having a base sequence corresponding to a 5′-terminal region in a base sequence shown in SEQ ID No. 1 of SEQUENCE LISTING, and
(2) oligonucleotides having a complementary base sequence to a 3′-terminal region in the base sequence shown in SEQ ID No. 1 of SEQUENCE LISTING.

7. The kit as claimed in claim 6, wherein the oligonucleotide primers comprise from 15 to 35 nucleotides.

8. The kit as claimed in claim 6, wherein the oligonucleotide primers are a pair of oligonucleotide primers selected from the group consisting of those shown in SEQ ID Nos. 2 to 8 of SEQUENCE LISTING, provided combinations of SEQ ID Nos. 2 and 4, 3 and 5, and 6 and 7 are excluded.


SEQUENCE LISTING (8 sequences)

SEQ ID NO: 1 - DNA, 12988 bp, Bovine
acgtcgttgatcctgtgggccgttttcaggtttgaagcttatctcggagccgaaaaggcagggcaccggcatggcgaaaaacatggctatcggcatcgacctgggcaccacctactcctgcgtaggggtgttccagcacggcaaggtggagatcatcgccaacgaccagggcaaccgcaccacccccagctacgtggccttcaccgataccgagcggctcatcggcgatgcggccaagaaccaggtggcgctgaacccgcagaacacggtgttcgacgcgaagcggctgatcggccgcaagttcggagacccggtggtgcagtcggacatgaagcactggcctttccgcgtcatcaacgacggagacaagcctaaggtgcaggtgagctacaaaggggagaccaaggcgttctacccggaggagatctcgtcgatggtgctgaccaagatgaaggagatcgccgaggcgtacctgggccacccggtgaccaacgcggtgatcaccgtgccggcctacttcaacgactcgcagcggcaggccaccaaggacgcgggggtgatcgcggggctgaacgtgctgaggatcatcaacgagcccacggccgccgccatcgcctacggcctggacaggacgggcaagggggagcgcaacgtgctcatctttgatctgggagggggcacgttcgacgtgtccatcctgacgatcgacgacggcatcttcgaggtgaaggccacggccggggacacgcacctgggcggggaggacttcgacaacaggctggtgaaccacttcgtggaggagttcaagaggaagcacaagaaggacatcagccagaacaagcgggccgtgaggcggctgcgcaccgcatgcgagcgggccaagagaaccttgtcgtccagcacccaggccagcctggagatcgactccctgttcgagggcatcgacttctacacgtccatcaccagggcgcggttcgaggagctgtgctccgacctgttccggagcaccctggagcccgtggagaaggcgctacgcgacgccaagctggacaaggcgcagatccacgacctggtcctggtggggggctccacccgcatccccaaggtgcagaagctgctgcaggacttcttcaacgggcgcgacctcaacaagagcatcaaccccgacgaggcggtggcgtacggggcggcggtgcaggcggccatcctgatgggggacaagtcggagaacgtgcaggacctgctgttgctggacgtggctcccctgtcgctgggactggagacggccggaggcgtgatgaccgccctgatcaagcgcaactccaccatccccacgaagcagacgcagatcttcaccacctactcggacaaccagccgggcgtgctgatccaggtgtacgagggcgagagggccatgacgcgggacaacaacctgctggggcgcttcgagctgagcggcatcccgccggccccgcggggggtgccccagatcgaggtgaccttcgacatcgacgccaatggcatcctgaacgtcacggccacggacaagagcacgggcaaggccaacaagatcaccatcaccaacgacaagggccggctgagcaaggaggagatcgagcgcatggtgcaggaggcggaaaagtacaaggcggaggacgaggtccagcgcgagagggtgtctgccaagaacgcgctggagtcgtacgccttcaacatgaagagcgccgtggaggatgaggggctgaagggcaagatcagcgaggcggacaagaagaaggtgctggacaagtgccaggaggtgatttcctggctggacgccaacaccttggcggagaaggacgagtttgagcacaagaggaaggagctggagcaggtgtgtaaccccatcatcagcagactgtaccagggggcgggcggccccggggctggcggctttggggctcagggccctaaagggggctctgggtctggccccaccattgaggaggtggattaggaatccttccctggattgctcatgtttgttatggagactgttgggatccaaggctttgcattgccttatatatcttcctttcatcagccatcagctatgcaagctgtttgagatgttgaactgtcccttttatgaaattaggaactcttttttccagagtcttaagtatagagctgaatgtatagtgccatcttttgtcagtttctttttgtagtattcatgccaaactcaagctatttttcacccgtttctgtttacttccaagtaaataaactcaaataattcgagtgatgtttgcttctgtgtttttattttgaagttagaaggatctgtagaggttgtctgttttacagtatccaaaaatgaactgcaattggcctcttagataaggtcagggatccagaaaagaatacagcattatgacacatttcttttaggcaaatagtatccttgggaaacataaagctgctcatttgaatggttgtgtttgtgaatccagaaaatgttaagggttactggcatggtagcctcaaggttgggcggggggtccatactttacgggtgaactcaaaaggtgcctgtagtggcagtattcctggagaagcaggcaaataagaggcagttagattggaagtcatgggtgctgctgcttgttagtacaggtgataccttagagccttgttacttaatctagattcagcatgaaagagaaggtgagtcctaaattggcactgaggaaatgtgaattctagtactggcttgcctaattatgcatgattgcgttagccactgtgatcctcaagtctcacagtttaaaatggaagggtttggcctgatgctaaagtttaatttcttaaaagaatgctgagataaaaatgctgcgtttccagtactggttacctacattttaagtatcccagtgagtaccttagagaggtgtcactgtttcatgccccagcaggaggacggacccccagtatttcagtgtgcttacctaccaggtactgtaccaggggccttttacatgtttattaattcccattccaccatattgagtataggcagtgtttggcttccacaggtggacgtatgtggagacttaaaaggcactggcttaaatttattacaagggtaaaaaaacgggttcagggaagatgttgaacctggattccaactgaggttttattgttttttgctctgctgcccacagggctttgtgcatgtctggttctgggtctaccctaggtttcacaatcggtaatctttctgctttgacaatgtataatcctaaacaactatgtcagataatacggttaatgctagaggtttaatactggttaatttagaagagtgattgaaaaaacctgcagcactgcaccaggaagccttaaccacaggcttccttcccctgcagatgcttcttgctttaactgttgctagaattctgggaagagtcccctccacagcctgtttgtgggaaaaggcctggcacaatcctcacgacttggggagtgagcccctttaaaaggcaattttatctggggattacagagattctggaaccaggtggaagtggtgattgcacaaactgggctagggaccactaaattctacactttaaaatggtttatgtgaattcaccaaaagtagtttttaaaaaaaaattgtgtcaacattctggaaaaacactttgtgagtgtgtgtatctcaaggcccaccaaatctttcactaaatacttgcattagaagaaactcttaatggtaataacatgtagaggtagacctgtccctgtaagtttggaaatggaaatctaagagatgcttagacttgcaggccagcatataaacacaggtttaatcctcagggtaggtgaactgtagcacggtggactgtagccacaatgtgagtcacccttcatggggatatgcggttggaacacgacctcctctacccccacagaactgcagtaccatctgtgactgtcatctgcagataatacaataactcttgaagcagtcaccctactttagggggaggtggcaagggatggggagggtggggtggagattgggaaagacctaacaaacacctttgataagagagattagggaaatctccagaaattaatttggagaaaatgagttcctatggctaaaccagttaagattatcagggtgttttattaggaagtcaatatataatgttactgcacagtcccttgcacagactactttgaaaataatcaccttcaacatgaagctgagggacaaagagaatgcaaagtcattcctggagaaggtgattgcggtagcagcaagaactcggggtgggggtgggggggaggaggtgcatcaaggaaaaataatggtcgatcaaaaagcatttttaaaatctaacaccttccctaattccaatctcacctacttccctatgccagccctgaaaaattagattgttatggtaatgtgactgattttaaatccaagatactacgttattaacacatagttactcctggtgtttaactggattctgtcattaaaaatgaaaaggataccaaagcaataacataattgtgagagaagtgcacagaagcatgggctttcagttaaaataaatggttttcaggtgaaaagtcaacactggcgatttctgagggggcgagcctcaaggtaggaataagaaagggcaactgtcatcattctttattccaactgatcaccttaaatccatccccaagggtcacccgcaaagtatccagtgcagttcagtaggatatagcaaccccatcagtcctctcctaactccagctcacgtagagacgttaaggggtcaggtatcgcagcgaattcgggatgccgagccaacctgccccaccccacgggcgccagtaccgcccagcaggaaatcggaggaaagggcacggcggggaaggagggagggcacacaggaaatacagggtaagggggcgggggagtccagaagatcagaatcaccccagaggatcttccacctttttacccgtccagacgtccccaggagagccagggactagattcgggagatgggacggcggcagagagaagacagcaagctcccagctgtagccaatccctgcccagggctgcggctcacccgcctctggcggtggggaccttctagcttctggcaaccccaatccatccgacttacttgtgtcagttacaaacctgtccagtgttttcacccaacatattagcgagtttgagggaaactctaaaggtctctcctttactgactcctttaatcccattttgaaaaagaaccgaagaacgccggcaccggccaggcaactccgcggccagccccgccgtcaggccccgccccgctccatcggggtcttactcgctctggctccttgcccccgtttcgggctgtgtcaggaactttctggagctctctgggctcagaggcggggactggctcgtaggaacactcttcaacaaacaaactgccccacccaagtctccctcccttcctctgttaacagccgaccagtctgtgataacgggaaggggagacggtcctgggagaacctggaagggccgaaaaggtggaagtgtgggtgttgtcgggggaagcggcggagctgggggtgcgtagataggcgtgagtcagaagcaacagcctggaggtgagtctccgcaggtcacacacccccatggtgcacgtagagccctggcattcactctttactgtcgtccatggttgtttctgttcttcttttatagagcgtggaacgatagggtttatgtgccagcattgagaggagtccaaagtagaaagtatgccgacatgttagttcaatcaccggttccgtaattacctgtctgggtgatctggccaagccacgaaacctctgaacctttgtgctcatctttgaaaacagaaaggtttggctgaaggactctgcctaaaaatctgaagatagtttttatggtaaaccgaaagtattactatcatagtcctggtagtaatccccaaccttgtaagcacctcagtaagaaatgattgagagatgagactcgagagagtgttacttcaataaaagaatgaagggcacaaacttttgagtacaactctgtcacagccactgaactagtcttttaaatattgtctttgtaatccttgatggtatcatactatgaaataaatattaattctaatttatacaacttgtgtaatttagttcatttacacgtacttcattgttaagaaagaaaaacagcttcaacaaggagatagagtccagatacaaacccaggtcttgcctttcccagttttttcccccatgctgctggaaattagcagagttcccaggcctttgccacacttccctggtggatcagagggtgaagaatctgcccacagtgcaagagacctgggttctatccctgagtagagaagatcccctggagaagggaatggcgacccactccagtgttcttgtgtggaaaatcccatgggcagaggagcctggccggctacagtccacggggtcacaaaggagtcggacatgactgggtgactaacactgtcaggcctttgccctttgaaggttacaaatgcctggctcagggctcgcctggtggctcatcggtaaagaatccgcctgccaatgcaggagacacaggttctattcctgatccaggaagattcccacatgtcctcgttccaaggagcagctaagcctgtgtgccacaactattgagcacgtacagcccattctctgaaacaagagaagccaccacaatgagaagcctgcttacccccaactcaactagagaatagcctctgctcaccacaactagagaaaagcctctgtagcagcagagatctagcacagccaaaaataaaatgaaaaaatgcctggctctaggtgtcacattgttctcttttgcttctgtctgaaaaacctagaattatactgtcttttaaaaacaaatagacttgagaaaaaccatactagatgaaaaactgtaggaaaaaggagagagaacaaaaaaagatcctgcaacttcagggtgaggacggctccccccgccccacccacttccttcccttggcagttagcattcttggcagtctctctcccatccccaacccttaaattttaccctgtcacccggtcaggcttgggcaaccttaatcttgattcttccaaacactaaacccgattttaaaaaactaattccaaaatgcatcaaataaagttgtgaaaagtctcttgggattcttaaaatctccttgctgctgctgctactaagtcgcttcagttgtgtccaactctgtgcaaccccacagacggaagcccaccaggctccccaatccctgggattctccaggcaagaacactggagtgggttgccatttccttctccaatgcatgaaagtgaaaagtgaaagtgaagttgctcaggagtccgactcttagcgaccccatggactgcagcctaccaggctcctccgttcatgggattttccaggcaagaacactggagtgggttgccattgccttctagagttacactattacactcattgatcatatatcgaactatacatttgatcaactgcttcaagtctagtcatcatttctgttgaaagctcagtcatatacttggtaatacaagaaataataatcttgtgaaacaagcaaaatacaaatggtatagttaataacattagtggaactaaaaggagatattttagccatgagcctcccacaccagttttttttaaagattgtcaagactagggaatgggtacttagagcagaaatctgatttttcatgtggttcaaatgtgttacattaaaggatttatcaggtacaaaaatacagcattcagtttgaattatagcacagctatctccctgagatgctgtcaagagtcttgcagttgtgtagcagggcctttctcattatagagatctcagaagtcaataggtgaatagcctgattatcatttaaagcttatgaaagttgttaaggcttagatatggtcaattacatcctccaaccccattgaaggcatgcacacgcgtgcgcacgcgcgcacacacacacacacacacacacacgctgctaaatggtcatacaccaaatctccttaggcaccaattaaaccggtacctgagttcctgccttgggaagtgtccagtgttaaaggaagacaaaattcaagagactctcctcataggaaatggaaaagaaatacggatatttaggtttccgggtcatccacagagagagacaacgcaaagtgtaggttaatacagtgtgtagctgactgcttgattcatgaaaaacagcattttcaagtggctcccccactcctccaccccagcaacagcaagatttgaggccctatcacctgtctccctgtcgagcagtggagacaatgatgccctttgcttcaagccaatagaggaagagaactgcaaattttggagaggagagcgaatccagaattcctgctggtagcagctgatgggggagaaggcaatggcaacccactccagtgttcttgcctggagaatcccagggacgggggagcctggtgggctgctgtctctggggtcgcacagagtcggacacaactgaagtgacttagcagtagcagcagcagctgatggtgaggaagacaggggagaggggatgaggttaaggacttctctggaggtgaacacttctctggaagtgttcacaaactgggtggctaagatggacgtttggggaatcccctttcagatactgcataaagagatggaaaattcctgaagtttaaccagtttgactagattaaggaggtgattcattggagagccacacctgaatgtaaaaaaagttatcacctacctgcacagtgaaagataaaaatattgctttaacaaatctgtatatctgattaacctgaacaaattataaaataaactgaataccctcagatttcaggaagaggtgtttgatgaatggctgtgcgcgcgcgcgcgcgtgtgtgtgtacgtgtgtaaacgtcagttaagcaaaagtgttcaaagcgagatttcttccctttatcagaaattgcctcctcaggtacttctctggtggtccagaagggctaagactctgtagaggagaatgcaggcggcctgggttcgatctctggtcaagaaaatagatcccacatgctacaactaagattgaccatgctacaactaaggcttagctattaattttaaaacaacaacaacaaaaccccacaactgcctcctccgacttgtgctgttatgttttctatgctcaagacatgtggatacagtaatgagtctatttcatgggttgtgaatcccctctactatggctttaatgtccctcacattttcactttaggtgcctaataagggatcttgcattgcccataaaggaagaagaaacaaaagccaaaataaattaccaaatgtcactgtatttaaaacaggaaggaggctaacaacagaaagctgaaatctaggataaaaagttaaatggacgaattaagtacacagcaaacaacctgaacttttagaggagatagaacctaggtcctgccaacctttctcaccttccagcatcattccagactgtttacaatgggccacccgccaaccaactatatagcatgctcttcaaacaggactgaacgctcccccacccccaccctcgcaggctcaccaccacaccacatttacttaaaagtagtggacagcctaggagccgcaaatgacaaggcagaagaccgaattcgggactcaggttaatccaggcaccactgatcatccgaggctgaaccaggaatttaaaaggcacagaggaggggaggggtgcgtccgcacctggggctgggaaagatgaggaatccggagaagcgcaaaggacagctaaatatctatggaaaatattttctttctcaagcccagtccagcccgaggagaaagggagcagctctgggcggggacaggggcgctgtggctccagccctgcccttcccacgctcccccgaccgagcaggtcccttctaaggcgttgggaaccttctacaatctaaaaaccatatacctaattgattttcttctgaaaattaaaatttcccctcccatctgaatagggctaaagaggagccaaaacttaaacagcttcaactctctccttttccttcccattttaaaaataagatgggaaaagcgccgcggatgaccaaggcatttctcggacagcccggccgctcggcgagccagcccaaacgtggctgcttccatcagcgttagcctccgatcactctccttggcccacagatagccaaccctcttcgagaaactcgggaactttctgtattttggctgtcccggcagtcgtgtagcccttaattctactttaaaccaccaaactaatttgagccccgagatcctctcaccgccctacaattaattacaagcccagggctgatccttccagtcgactccaaactacttggctggctggtcgccaggaaaccagagacagagtgggtggaccttcccagcccctctccccctctccttaggactcctgtttcctccagcgaatcctagaagagtctggagagttctgggaggagaggcatccagggcgctgattggttccagaaagccagggggcaggacttgaggcgaaacccctggaatattcccgacctggcagccccactgagctcggtcattggctgacgaagggaaaaggcggcggggcttgatgaagaattataaacacagagccgcctgaggagaaacagcagcctggagagagctgataaaacttacggcttagtccgtgagagcagcttccgcagacccgctatctccaaggaccgccccgaggggcaccagagcgttcagttttcgggttccgaaaagcccgagcttctcgtcgcagatcctcttcaccgatttcaggtttgaagcttatctcggagccggaaaagcagggcaccggcatggcgaaaaacacagctatcggcatcgacctgggcaccacctactcctgcgtaggggtgttccagcacggcaaggtggagatcatcgccaacgaccagggcaaccgcaccacccccagctacgtggccttcaccgataccgagcggctcatcggagatgcggccaagaaccaggtggcgctgaacccgcagaacacggtgttcgacgcgaagcggctgatcggccgcaagttcggagacccggtggtgcagtcggacatgaagcactggcctttccgcgtcatcaacgacggagacaagcctaaggtgcaggtgagctacaagggggagaccaaggcgttctacccggaggagatctcgtcgatggtgctgaccaagatgaaggagatcgccgaggcgtacctgggccacccggtgaccaacgcggtgatcaccgtgccggcctacttcaacgactcgcagcggcaggccaccaaggacgcgggggtgatcgcggggctgaacgtgctgaggatcatcaacgagcccacggccgccgccatcgcctacggcctggacaggacgggcaagggggagcgcaacgtgctcatctttgatctgggagggggcacgttcgacgtgtccatcctgacgatcgacgacggcatcttcgaggtgaaggccacggccggggacacgcacctgggcggggaggacttcgacaacaggctggtgaaccacttcgtggaggagttcaagaggaagcacaagaaggacatcagccagaacaagcgggccgtgaggcggctgcgcaccgcatgcgagcgggccaagagaaccttgtcgtccagcacccaggccagcctggagatcgactccctgttcgagggcatcgacttctacacgtccatcaccagggcgcggttcgaggagctgtgctccgacctgttccggagcaccctggagcccgtggagaaggcgctacgcgacgccaagctggacaaggcgcagatccacgacctggtcctggtggggggctccacccgcatccccaaggtgcagaagctgctgcaggacttcttcaacgggcgcgacctcaacaagagcatcaaccccgacgaggcggtggcgtacggggcggcggtgcaggcggccatcctgatgggggacaagtcggagaacgtgcaggacctgctgttgctggacgtggctcccctgtcgctgggactggagacggccggaggcgtgatgaccgccctgatcaagcgcaactccaccatccccacgaagcagacgcagatcttcaccacctactcggacaaccagccgggcgtgctgatccaggtgtacgagggcgagagggccatgacgcgggacaacaacctgctggggcgcttcgagctgagcggcatcccgccggccccgcggggggtgccccagatcgaggtgaccttcgacatcgacgccaatggcatcctgaacgtcacggccacggacaagagcacgggcaaggccaacaagatcaccatcaccaacgacaagggccggctgagcaaggaggagatcgagcgcatggtgcaggaggcggaaaagtacaaggcggaggacgaggtccagcgcgagagggtgtctgccaagaacgcgctggagtcgtacgccttcaacatgaagagcgccgtggaggatgaggggctgaagggcaagatcagcgaggcggacaagaagaaggtgctggacaagtgccaggaggtgatttcctggctggacgccaacaccttggcggagaaggacgagtttgagcacaagaggaaggagctggagcaggtgtgtaaccccatcatcagcagactgtaccagggggcgggcggccccggggctggcggctttggggctcagggccctaaagggggctctgggtctggccccaccattgaggaggtggactaggggccttactttttgtctgtctgtagtagacc

SEQ ID NO: 2 - DNA, 20 bp, Artificial Sequence
  Oligonucleotide to act as a primer for PCR
aaccccatcatcagcagact

SEQ ID NO: 3 - DNA, 21 bp, Artificial Sequence
  Oligonucleotide to act as a primer for PCR
cacagaagcaaacatcactcg

SEQ ID NO: 4 - DNA, 20 bp, Artificial Sequence
  Oligonucleotide to act as a primer for PCR
gcattgcccataaaggaaga

SEQ ID NO: 5 - DNA, 20 bp, Artificial Sequence
  Oligonucleotide to act as a primer for PCR
tggaaggtgagaaaggttgg

SEQ ID NO: 6 - DNA, 19 bp, Artificial Sequence
  Oligonucleotide to act as a primer for PCR
acgtcgttgatcctgtggg

SEQ ID NO: 7 - DNA, 19 bp, Artificial Sequence
  Oligonucleotide to act as a primer for PCR
tatctcggagccgaaaagg

SEQ ID NO: 8 - DNA, 29 bp, Artificial Sequence
  Oligonucleotide to act as a primer for PCR
ggtctactacagacagacaaaaagtaagg